(19) United States
(12) Patent Application Publication
(10) Pub. No.: US 2005/0106718 A1
(43) Pub. Date: May 19, 2005

(54) Cell scraper and method of using same

(76) Inventors: Sundaravadivel Balasubramanian, West Haven, CT (US); Dhandapani Kuppuswamy, Charleston, SC (US)

Correspondence Address: B. Craig Killough;Barnwell Whaley Patterson & Helms, LLC, P.O. Drawer H, Charleston, SC, 29402-0197, US

(21) Appl. No.: 10/991,253

(22) Filed: Nov. 17, 2004

Related U.S. Application Data
(60) Provisional application No. 60/520,872, filed on Nov. 18, 2003.

(51) Int. Cl. C12M 1/26

(52) U.S. Cl. 435/309.1; 435/283.1; 422/100

(57) ABSTRACT

A pipette tip has a blade formed along a forward end of the tip that functions as a cell scraper. The device is constructed to allow the tip to be articulated so that the blade contacts the culture plate's surface parallel to, or on a slanting angle relative to, the horizontal surface of the plate, when the pipette is held vertically. The bent tip may be used to pipette the solution, in addition to scraping the cells. After scraping, the tip of the invention can be used to collect the cell suspension from the culture dish.

Applicant claims the benefit of U.S. Provisional Application Ser. No. 60/520,872 filed Nov. 18, 2003.

### FIELD OF THE INVENTION

This invention relates generally to the lysis of cell cultures for analysis, is more specifically directed to a device for the disruption of culture cells, and a method of using the device.

### BACKGROUND OF THE INVENTION

Most biological experiments with adherent cell cultures involve the lysis of the cells for analysis. The lysis of cells from a monolayer of cells culture on a tissue culture dish is normally performed at or near the conclusion of an experiment with the cells. This process is performed by adding a desired lysis solution to the cell monolayer, using a micro-pipette tip. The cells are subsequently scraped using a cell scraper. The cell suspension is then collected using a micro-pipette tip, and used for biochemical analysis. In the prior art, the cell scraper and micro-pipette tip are separate devices, and the process is performed using these separate devices.

### SUMMARY OF THE PRESENT INVENTION

The pipette tip of the present invention comprises a blade formed along a forward portion of the tip that functions as a cell scraper. The device may be used with a pipette. The device is configured to allow the tip to be articulated so that the blade contacts the culture plate's surface parallel to, or on a slanting angle relative to, the horizontal surface of the plate, when the pipette is held vertically. The bent tip also can be used to pipette the solution, in addition to scraping the cells. After scraping, the tip of the invention can be used to collect the cell suspension from the culture dish. The cell scraping pipette tip can be used to (i) withdraw solutions in both the straight and articulated forms and (ii) scrape adherent cells when the tip is articulated from the vertical, exposing the blade to the surface of the adherent cells. The cell scraping pipette tip according to the invention is more convenient to use than the conventional combined usage of pipette tip and cell scraper. Further, this invention allows the user to handle only one device to complete the cell scraping process including dispensing the lysis solution, scraping cells and collecting the cell suspension.

### DESCRIPTION OF THE DRAWINGS

FIG. 1 is a perspective view of the cell scraper of the present invention.

FIG. 2 is a perspective view of the cell scraper shown in FIG. 1, with the tip of the cell scraper articulated at an angle relative to the remainder of the device.

FIG. 3 demonstrates a method of using the device in conjunction with a pipette to apply a solution to adherent cells.

FIG. 4 demonstrates a method of using the device with the tip of the cell scraper articulated and contacting cells on the surface of a culture dish.

FIG. 5 shows the device being used to collect the cell suspension from the culture dish.

FIG. 6 is an additional embodiment of the cell scraper.

FIG. 7 is a perspective view of the cell scraper shown in FIG. 6.

### DESCRIPTION OF THE PREFERRED EMBODIMENTS

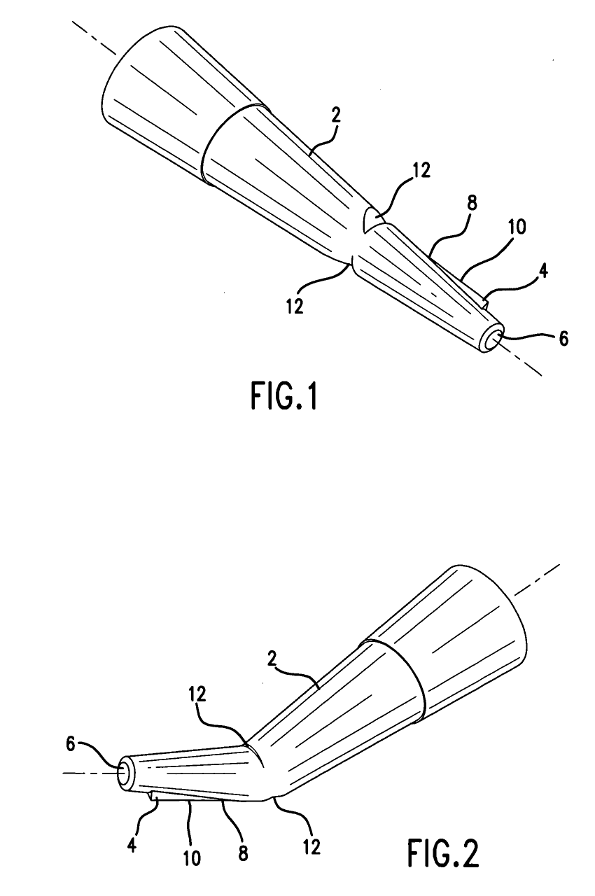

Referring now to the drawing figures, FIG. 1 shows a micro-pipette tip 2 according to the present invention. The micro-pipette tip has a blade 4 formed along the tip. In the embodiment shown, the pipette tip tapers from a larger diameter to a smaller diameter at the forward opening 6 of the tip. The blade is formed as a unitary member with the micro-pipette tip. The blade is preferred to extend from one side of the forward portion of the micro-pipette tip, and outwardly from the forward portion of the micro-pipette tip.

To account for the tapering of the micro-pipette tip, the blade is tapered in the opposite direction. The blade is formed to have a larger dimension at the most forward portion of the blade, while tapering to a smaller point 8 on the micro-pipette tip. In the preferred embodiment, the edge 10 of the blade is generally parallel to a center line of the micro-pipette tip.

The blade may be formed with the pipette tip as a unit, wherein the blade is an integral part of the pipette tip. The pipette tip may be formed of plastics that are commonly used to form pipette tips that are in current use. The use of this plastic allows the device to be made at low cost, yielding a micro-pipette tip that is disposable after a single use.

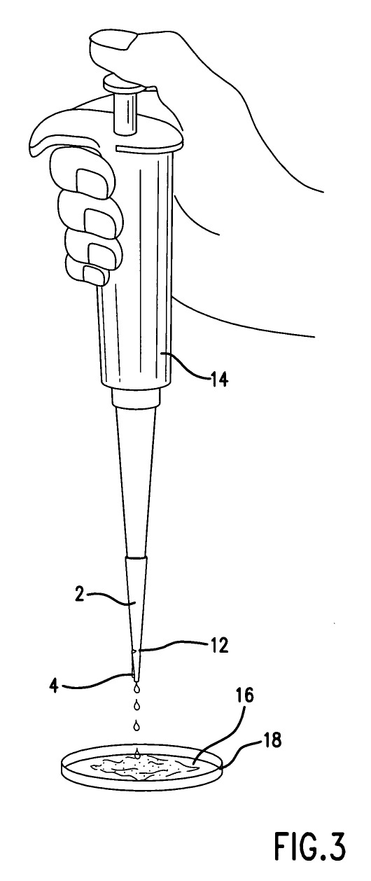

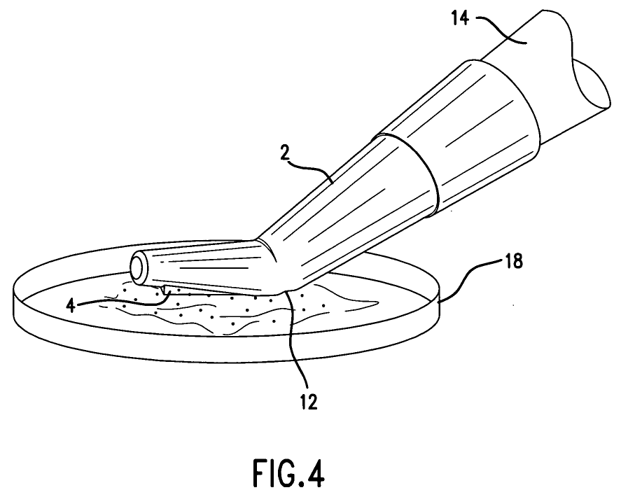

The micro-pipette tip is adapted to fit on the pipettes that are commonly known and used. The micro-pipette tip is generally hollow and frusto conical in shape, and it is formed in steps or sections. The hollow body accepts the end of the pipette, when the micro-pipette tip is mounted as showed in FIGS. 3 through 5. The hollow body of the device allows the device to be mounted to a pipette, and also allows fluids to pass though the tip and opening 6.

In the preferred embodiment, the device has a hinge 12 that is located between the blade and the pipette. As shown in the drawing figures, the hinge is positioned at approximately one-fourth of the length of the micro-pipette tip from the forward opening, but may be placed in the forward half of the micro-pipette tip. The hinge allows the blade to be articulated at an angle relative to the centerline of the tip 2, as shown in FIG. 2. By positioning the hinge within the forward half of the micro-pipette tip, there is adequate room for the to pipette tip to accommodate the pipette within the tip, without interfering with the operation of the hinge.

In the preferred embodiment shown FIG. 1, the hinge 12 is provided by reducing the amount of material that is present where the micro-pipette tip bends. The hinge provides sufficient flexibility to permit the forward tip of the device to be articulated as shown in FIG. 2 or FIG. 4 by the application of manual pressure to the tip, or by pressing against the pipette while the forward tip is held against an object. It is preferred that the tip be capable of articulation relative to the center line of the device up to at least 60 degrees. The forward tip of the device may be articulated at an angle greater than 60 degrees, however, if the angle is too great, it will interfere with fluid flow through the tip. In many processes in which the tip is used, it is not necessary to induce fluid flow through the tip while the forward end of the device is articulated and is being used as a scraper.

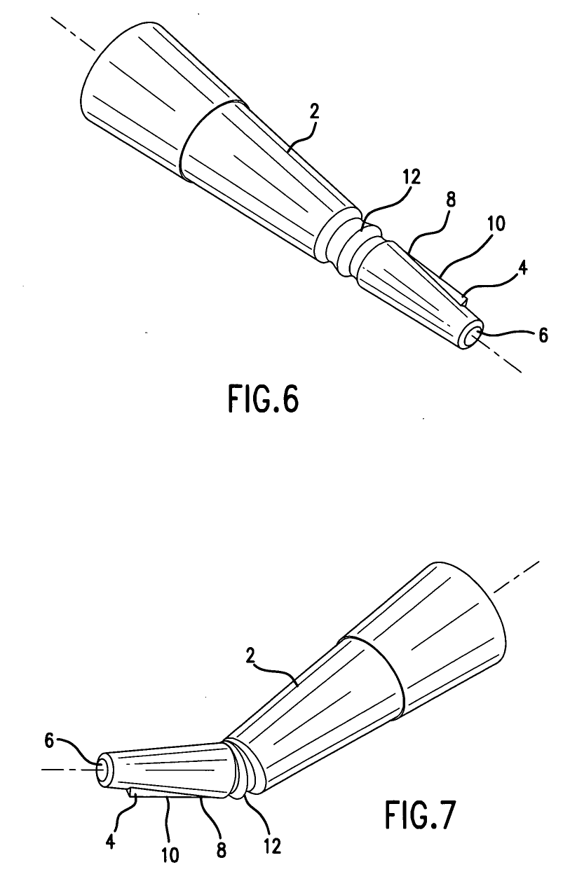

In the embodiment shown in FIG. 6 and FIG. 7, the hinge is formed of peaks and valleys that are present in the micro-pipette tip. The hinge comprises multiple areas of increased and decreased dimension that circumscribe the tip, so that the forward section of the micro-pipette tip that comprises the blade can be bent or articulated relative to the remainder of the tip. This structure is similar to that found in flexible plastic drinking straws, but is provided with additional resistance, and is constructed so that the forward portion of the tip that comprises the blade will not materially move or deform after positioning, and while the device is being used to scrape cells from a culture dish.

In one embodiment, the device is used with a 200 μl micro-pipette tip. The blade may have a length of 6.5 to 8.5 mm and a thickness of 0.10-0.14 mm. The blade starts about 1.5-2.5 mm away from the tip's tapered end with a height of 0.5-0.6 mm. It is extended for 6.5-8.5 mm toward the broad end of the micro-pipette tip where it has a height of 0.15-0.25 mm. The thickness, height and length of the blade can be varied. This can be scaled up or down for other pipette tips (for example 100 μl) depending on the need. The blade may extend for up to ninety (90%) percent of the length of the micro-pipette tip, while not extending completely to the forward end of the tip.

The device may have two hinge points (or pits). These hinge points are intended to help the user in bending the tip by applying a small force at the tip of the pipette so that the blade can contact the culture plate's surface parallel to the ground when the pipette in held vertically.

Referring again to the drawings, as shown in FIGS. 3 through 5, the device is used by mounting the micro-pipette tip 2 comprising the blade to a pipette 14, or perhaps, to a syringe. The device is used in a conventional manner to apply a lysis solution to cells 16 that are adhered to a culture dish 18. FIG. 3.

After applying the solution to the adherent cells, the forward tip of the device is articulated at an angle relative to the center line of the pipette. The micro-pipette tip is articulated so that the blade is in the position shown in FIG. 4, and the blade is used for cell lysis. The end of the micro-pipette tip comprising the blade is repositioned relative to the remainder of the micro-pipette tip by applying pressure to the tip, such as pressing the tip against the culture dish. The reduced amount of material that forms the hinge will allow this end of the micro-pipette tip to be deformed relative to the pipette so that the blade is in position for use.

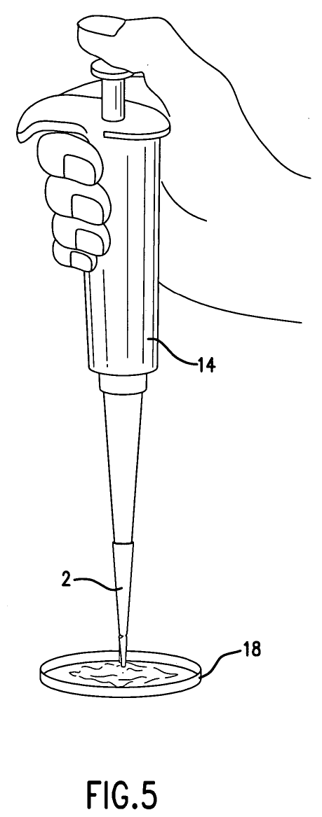

After the blade from the culture dish scrapes the cells, the pipette may be used to remove the cells, which are now suspended in a liquid. The forward end of the tip is articulated so that its centerline is approximately in line with the centerline of the remainder of the tip, and the suspended cells are withdrawn into the pipette. The realignment of the end of the micro-pipette tip with the centerline generally only needs to be approximate in order to allow the cells to be drawn into the pipette, and is usually sufficient if the lumen that is present in the micro-pipette tip is not materially blocked. Realignment may be performed by appropriate pressure against the tip, such as by pressing the tip against the side of the culture dish. The cells may then be eliminated from the pipette at a remote location, and used as desired. FIG. 5.

Cell scraping can be performed either to prepare the cell extract or to make a live cell suspension by scraping the monolayer for passaging the cells. The cell scraping pipette tip is more convenient to use than the conventional combined usage of micro-pipette tip and cell scraper. This cell scraping pipette tip can be produced for less cost than the existing combination of devices. Further, the cell sample loss is drastically reduced. The invention allows the user to handle only one device to complete the cell scraping process including dispensing the lysis solution, scraping cells and collecting the cell suspension. The existing technology needs interchanging between pipette tip and cell scraper. These qualities make the invention very useful in biological laboratories engaged in adherent cell culture experiments.

1. A pipette tip, comprising a generally hollow body, said generally hollow body having a blade formed on an exterior thereof, and said hollow body having an orifice in a distal end thereof.

2. A pipette tip as described in claim 1, wherein said generally hollow body tapers from a larger proximal end toward said distal end that is smaller than said larger proximal end, and wherein said blade is generally wedge shaped and tapers from said distal end toward said larger proximal end.

3. A pipette tip as described in claim 1, wherein a longitudinal edge of said blade is generally parallel to a longitudinal centerline of said pipette tip.

4. A pipette tip as described in claim 1, wherein said generally hollow body comprises at least one hinge, and wherein a distal portion of said generally hollow body articulates relative to a remainder of said generally hollow body at said at least one hinge.

5. A pipette tip as described in claim 4, wherein said at least one hinge is formed between said blade and a proximal end of said generally hollow body.

6. A pipette tip as described in claim 5, wherein said generally hollow body comprises an additional hinge that is formed between said at least one hinge and said proximal end of said generally hollow body.

7. A pipette tip as described in claim 1, wherein said generally hollow body and said blade are formed as a unitary member.

8. A pipette tip as described in claim 7, wherein said generally hollow body and said blade are formed of plastic.

9. A pipette tip as described in claim 4, wherein said at least one hinge is formed by a reduced wall thickness of said generally hollow body.

10. A method of using a pipette tip as described in claim 4, comprising the steps of
mounting said generally hollow body to a liquid source;
applying a liquid from said liquid source to an object through said orifice of said generally hollow body;
articulating said distal portion of said generally hollow body relative to a remainder of said hollow body; and
removing a material from said object by scraping with said blade.
